SEQUENCE LISTING (4 sequences)

SEQ ID NO: 1 - DNA, 20 bp, Artificial Sequence
  Synthetic DNA
acaataaaactttgctgcca

SEQ ID NO: 2 - DNA, 20 bp, Artificial Sequence
  Synthetic DNA
cctttttggacttcaggtgg

SEQ ID NO: 3 - DNA, 35 bp, Artificial Sequence
  Synthetic DNA
acaataaaactttgctgccatttagcttccttagc

SEQ ID NO: 4 - DNA, 35 bp, Artificial Sequence
  Synthetic DNA
cctttttggacttcaggtggatgtcaggctccctt

(19) United States
(12) Patent Application Publication
(10) Pub. No.: US 2006/0127978 A1
(43) Pub. Date: Jun. 15, 2006

(54) Nucleic acid recombination method, host cell and expression vector

(75) Inventors: Yasushi Shigemori, Kisarazu-shi (JP); Takehiko Shibata, Wako-shi (JP); Tsutomu Mikawa, Wako-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: AISIN SEIKI KABUSHIKI KAISHA, Kariya-shi (JP); RIKEN, Wako-shi (JP)

(21) Appl. No.: 11/071,331

(22) Filed: Mar. 4, 2005

(30) Foreign Application Priority Data
Mar. 4, 2004 (JP) 2004-060878

(51) Int. Cl. C12N 15/09; C12P 21/06

(52) U.S. Cl. 435/69.1; 435/320.1; 435/325

(57) ABSTRACT

This invention is to provide a nucleic acid recombination method which enables to carry out precisely homologous recombination of a desired target region, a host cell which enables to carry out precisely homologous recombination of a desired target region, and an expression vector which can be used for the nucleic acid recombination method and the host cell. The nucleic acid recombination method to carry out homologous recombination of a nucleic acid in a host cell employs a host cell which enables the expression and recombination of a RecA-like protein, and enables the expression of a RecX-like protein. And, the nucleic acid recombination method comprises a recombination step of a nucleic acid to carry out homologous recombination of the nucleic acid by the RecA-like protein expressed in the host cell, and a RecA inhibition step to inhibit the activity of the RecA-like protein by expressing the RecX-like protein.

### CROSS REFERENCE TO RELATED APPLICATIONS

This application is based on and claims priority under 35 U.S.C. §119 with respect to Japanese Patent Application No. 2004-060878 filed on Mar. 4, 2004, the entire content of which is incorporated herein by reference.

### FIELD OF THE INVENTION

The present invention relates to a nucleic acid recombination method to carry out nucleic acid recombination in a cell, a host cell which enables nucleic acid recombination in its own cell, and an expression vector which can be used for the nucleic acid recombination method and the host cell, and specifically, a nucleic acid recombination method to carry out homologous recombination of nucleic acids, a host cell which enables homologous recombination of nucleic acids, and an expression vector which can be used for the nucleic acid recombination method and the host cell.

### BACKGROUND

Conventionally, various methods have been proposed for cloning a target region such as a gene and a promoter region into a vector.

For example, Non-Patent Document 1 discloses a cloning method using a restriction enzyme. In this method, as outlined in FIG. 8, a vector is cleaved with a suitable restriction enzyme, and further a DNA having a target region for cloning (represented as Insert in the figure) is cleaved with a suitable restriction enzyme. Then, ligation of the target region and the vector is carried out by a ligase, to form a recombinant DNA. Then, the recombinant DNA is introduced into E. coli (transformation).

Further, Non-Patent Document 2 discloses a cloning method using a PCR reaction. In this method, as outlined in FIG. 9, a vector is cleaved with a suitable restriction enzyme. On the other hand, a target region for cloning (represented as Insert PCR product in the figure) is amplified using a PCR reaction. Then, ligation of the target region and the vector is carried out by a ligase, to form a recombinant DNA. Then, the recombinant DNA is introduced into E. coli (transformation).

Further, Non-Patent Document 3 discloses a cloning method using homologous recombination of nucleic acids. In this method, as outlined in FIG. 10, homologous sequences (Homologous sequence A and Homologous sequence B) which are homologous to the sequences of both ends (Sequence A and Sequence B) of a target region for cloning (represented as Insert in the figure) are inserted into vectors, respectively. In the present specification, the “homologous recombination region” refers to Homologous sequence A and Homologous sequence B, and, when these sequences are inserted into a region as shown in the figure, to the region which contains this region. Then, the DNA which contains the target region and the vector are introduced into E. coli, and homologous recombination is conducted between the target region and the homologous recombinant region of the vector by a RecA protein, etc. in E. coli, thereby the target region instead of the homologous recombinant region is incorporated into the vector to form a recombinant DNA.

[Non-Patent Document 1] Joseph Sambrook & Davis W. Russel (2001) Molecular Cloning: A Laboratory Manual, third edition. Cold Spring Harbor laboratory Press. Cold Spring Harbour, NY, pp 1.84-1.87 [Non-Patent Document 2] In vivo cloning of PCR products in E. coli. Nucleic Acids Research 1993, Vol. 21, No. 22, 5192-5197 [Non-Patent Document 3] DNA cloning by homologous recombination in Escherichia coli. Nature Biotechnology 2000 December Vol. 18 1314-1317

However, these conventional cloning methods each have problems.

In the method disclosed in Non-Patent Document 1, if the target region for cloning has a cleavage site for the restriction enzyme that is to be used, such target region is cleaved by the restriction enzyme so that the target region cannot be cloned as a whole. Especially, when the target region for cloning is long, a restriction enzyme which does not cleave the inside of the target region may not be found in many cases, so that it is hard to clone the whole target region. Also, even when the whole target region can be cloned by selecting a suitable restriction enzyme, in general, the upstream region and the downstream region are also cloned together in addition to the target region for cloning, and thus, it is not possible to clone only the target region without excess or deficiency. For example, even when only the region of a gene or only the promoter region is to be cloned, problems in that both of the gene and the promoter region are cloned, etc. may occur.

On the other hand, in the method disclosed in Non-Patent Document 2, it is possible to clone only the target region without excess or deficiency because PCR is used. However, due to the nature of the PCR reaction, mutation may occur at the target region for cloning by a reaction error. Then, problems may occur such that, for example, it is impossible to express a precise protein when expression analysis for the cloned gene is carried out. Further, problems may occur such that, for example, it is impossible to determine a precise base sequence when the base sequence of the cloned target region is sequenced.

On the other hand, the method disclosed in Non-Patent Document 3 can solve the problems of the methods disclosed in Non-Patent Documents 1 and 2. However, this cloning method is not yet put to practical use at present. The main reason is that not only the recombination between desired DNA regions, but also the recombination between undesired DNA regions may occur frequently because of recombination ability of E. coli, whereby the target region in its intact state cannot be cloned into the vector. In addition, this method has a disadvantage of very poor cloning efficiency.

In light of such circumstances, an object of the present invention is to provide a nucleic acid recombination method which enables to carry out precisely homologous recombination of a desired target region in a host cell, a host cell which enables to carry out precisely homologous recombination of a desired target region in its own cell, and an expression vector which can be used for the nucleic acid recombination method and the host cell.

### SUMMARY OF THE INVENTION

Means for solving such problems is a nucleic acid recombination method to carry out homologous recombination of nucleic acids in a host cell, wherein the host cell is constituted to be able to express a RecA-like protein with ability of recombination and to express a RecX-like protein, wherein the nucleic acid recombination method comprises a nucleic acid recombination step to carry out homologous recombination of the nucleic acids by the RecA-like protein expressed in the host cell, and a RecA inhibition step to inhibit the activity of the RecA-like protein by expressing the RecX-like protein.

In the conventional cloning methods using homologous recombination, the reason why the recombination between undesired DNA regions may also occur frequently is considered to be that a RecA protein works actively, whereby undesired recombination is promoted. Accordingly, it is considered that if the activity of the RecA protein can be controlled freely, i.e., if the activity of the RecA protein can be used only when it is needed, only the recombination between desired DNA regions can be carried out while suppressing the recombination between undesired DNA regions.

On the other hand, recently, it has been shown that a protein called RecX protein, of which functions and properties were unknown, has properties of inhibiting the activity of the RecA protein (for detail, refer to Escherichia coli RecX inhibits recA recombinase and coprotease activities in vitro and in vivo, J. Biol. Chem., 2003 Jan. 24:278(4): 2278-2285).

Thus, the present inventors have studied extensively on controlling the activity of the RecA protein by arbitrarily controlling the expression of the RecX protein in a host cell. As a result, the present inventors have developed a method to carry out recombination between desired DNA regions only while suppressing recombination between undesired DNA regions, thus have completed the present invention.

Thus, the present invention uses a host cell which is constituted to be able to express a RecA-like protein with ability of recombination and to express a RecX-like protein. Further, a nucleic acid recombination method of the present invention comprises a nucleic acid recombination step to carry out the homologous recombination of nucleic acids by a RecA-like protein expressed in a host cell, and a RecA inhibition step to inhibit the activity of the RecA-like protein by expressing the RecX-like protein.

In such nucleic acid recombination method, when a RecA-like protein is needed, the RecA-like protein can be used, and when the RecA-like protein is not needed, its activity can be inhibited by a RecX-like protein, whereby it is possible to carry out the recombination between desired nucleic acid regions only while suppressing the recombination between undesired nucleic acid regions. Accordingly, it is possible to carry out precisely homologous recombination of a desired target region in a host cell. Further, it is also possible to improve recombination efficiency as compared with that of the conventional methods although the reason is not clearly understood.

Therefore, for example, if such nucleic acid recombination method is used in cloning, it is possible to clone the target region for cloning in its intact state into a vector. Of course, in such nucleic acid recombination method, it is also possible to solve the problems of the method disclosed in Non-Patent Document 1, i.e., the problem in that the target region cannot be cloned as a whole, etc., and the problems of the method disclosed in Non-Patent Document 2, i.e., the problem in that mutation may occur at the target region, etc.

Here, the “host cell” is not limited as long as it is constituted to be able to express a RecA-like protein with ability of recombination and to express a RecX-like protein. Accordingly, the host cell may be a prokaryotic cell or a eukaryotic cell. For example, the host cell includes Escherichia coli, Saccharomyces cerevisiae, Bacillus subtilis, animal or plant cells, and the like.

The “RecA-like protein” is not limited as long as it has a function similar to that of the RecA protein of E. coli in homologous recombination. Accordingly, it may be a RecA-like protein of a prokaryotic organism or a RecA-like protein of a eukaryotic organism. For example, it includes a RecA-like protein derived from a prokaryotic organism which has high homology to the RecA protein, a RecA-like protein such as Rad51 and Dmcl found in a yeast and the like. Further, it may be a natural RecA-like protein or a modified protein obtained by modification of the RecA-like protein. Such modified protein includes a gene product made by inducing site-specific mutation, etc., in a gene which encodes the RecA-like protein, wherein the gene product comprises an amino acid sequence with deletion, substitution or addition of one or more amino acids, and further has a function similar to that of the RecA-like protein. Further, it may be a protein fragment of the RecA-like protein, wherein the protein fragment has a function similar to that of the RecA-like protein (a RecA-like protein fragment). Also, it is known that the RecA protein of E. coli is a key enzyme in homologous recombination, and is a protein which catalyzes pairing and strand exchange between DNA molecules having homologous regions, and functions on both of DNA modification and DNA recombination.

Further, the RecA-like protein gene which expresses the RecA-like protein may exist on the genome of a host cell, or on a vector such as a plasmid, a phage, a cosmid and the like. Further, the RecA-like protein gene is not necessarily originally contained in the host cell, but may be an exogenous gene. For example, it may be prepared by introducing a RecA-like protein gene derived from cells other than E. coli into E. coli.

The “RecX-like protein” is not limited as long as it has a function similar to that of the RecX protein of E. coli from the viewpoint that it inhibits the activity of a RecA-like protein. Accordingly, it may be a RecX-like protein of a prokaryotic organism or a RecX-like protein of a eukaryotic organism. For example, it includes a RecX-like protein derived from a prokaryotic organism, wherein the RecX-like protein has high homology to the RecX protein. Further, it may be a natural RecX-like protein or a modified protein obtained by modification of the RecX-like protein. Such modified protein includes a gene product made by inducing site-specific mutation, etc., in a gene which encodes the RecX-like protein, wherein the gene product comprises an amino acid sequence with deletion, substitution or addition of one or more amino acids, and further has a function similar to that of the RecX-like protein. Further, it may be a protein fragment of the RecX-like protein, wherein the protein fragment has a function similar to that of the RecX-like protein (a RecX-like protein fragment).

Further, the RecX-like protein gene which expresses a RecX-like protein may exist on the genome of a host cell, or on a vector such as a plasmid, a phage, a cosmid and the like. Further, the RecX-like protein gene is not necessarily originally contained in the host cell, but may be an exogenous gene. For example, it may be prepared by introducing a RecX-like protein gene derived from cells other than E. coli into E. coli.

The “nucleic acid recombination step” refers to, as described above, a step to carry out homologous recombination of nucleic acids by a RecA-like protein expressed in a cell. The RecA-like protein may be expressed at least at the time of this step. It may be expressed constantly in a host cell, or may be expressed at the time of this step, even if not constantly. For example, if a host cell originally expresses the RecA-like protein constantly, the gene of the host cell can be used as it is for the RecA-like protein gene. Further, a suitable inducible promoter may be attached to the RecA-like protein gene. In this way, it is possible to induce the RecA-like protein gene by inducing the host cell to express the RecA-like protein.

The “RecA inhibition step” refers to, as described above, a step to inhibit the activity of the RecA-like protein by expressing the RecX-like protein. The RecX-like protein may be made to be expressed at this step. For example, it is considered possible to attach a suitable inducible promoter to the RecX-like protein gene. In this way, it is possible to induce the RecX-like protein gene by inducing the host cell to express the RecX-like protein.

Further, the “homologous recombination of nucleic acids” in the present invention can be carried out by introducing a target nucleic acid which contains a target region for recombination and a recombination vector for incorporation of the target region into a host cell, and by performing recombination between them. Further, the recombination can be also carried out by introducing a target nucleic acid which contains a target region for recombination, and by performing recombination between the target nucleic acid and the genome of the host cell. Further, the recombination can be also carried out between different regions in the genome of the host cell.

Furthermore, in the above-mentioned nucleic acid recombination method, the method is preferably a nucleic acid recombination method, wherein the above-mentioned RecA-like protein is the RecA protein of Escherichia coli.

As described above, the RecA-like protein is not limited as long as it has a function similar to that of the RecA protein of E. coli in homologous recombination. However, the RecA protein is most preferably the RecA protein of E. coli from the viewpoint that the RecA protein gene which encodes the RecA protein of E. coli is easily available, and has been most studied on its functions and properties, and so on.

Furthermore, in any of the above-mentioned nucleic acid recombination methods, the method is preferably is a nucleic acid recombination method, wherein the above-mentioned RecX-like protein is the RecX protein of Escherichia coli.

As described above, the RecX-like protein is not limited as long as it has a function similar to that of the RecX protein of E. coli from the viewpoint that it inhibits the activity of the RecA-like protein. However, the RecX-like protein is most preferably the RecX protein of E. coli from the viewpoint that the RecX protein gene which encodes the RecX protein of E. coli is easily available, and that RecX-like proteins other than the RecX protein of E. coli have not been much studied in detail on their functions and properties at present.

Furthermore, in any of the above-mentioned nucleic acid recombination methods, the method is preferably a nucleic acid recombination method, wherein the host cell is Escherichia coli.

As described above, the host cell is not limited as long as it is constituted to be able to express a RecA-like protein with ability of recombination and to express a RecX-like protein. However, the host cell is most preferably E. coli in view of easy availability and easy handling. Especially, when cloning is carried out using the nucleic acid recombination method of the present invention, E. coli is suitable in view of cloning efficiency, ease of subsequent analysis and the like.

Furthermore, in any of the above-mentioned nucleic acid recombination methods, the method is preferably a nucleic acid recombination method wherein the host cell is prepared from a cell originally having a RecA-like protein gene which expresses the above-mentioned RecA-like protein in its own genome.

As described above, the RecA-like protein is not necessarily expressed constantly as long as it is expressed at least at the nucleic acid recombination step. Since the RecA-like protein gene which originally exists in the genome of the host cell usually expresses the RecA-like protein constantly, such RecA-like protein gene can be used as it is. As such, if the RecA-like protein gene originally contained in its own genome is used, labors for constructing a host cell may be saved since there is no particular need to transform the host cell for the RecA-like protein gene.

Furthermore, in any of the above-mentioned nucleic acid recombination methods, the method is preferably a nucleic acid recombination method, wherein the host cell has an expression vector into which a RecX-like protein gene is inserted, and in the above-mentioned RecA inhibition step, the RecX-like protein is expressed from the RecX-like protein gene.

As described above, the RecX-like protein should be controlled to be expressed at the RecA inhibition step. On the other hand, the RecX-like protein gene originally contained in the host cell is in general not externally controllable for its expression. Accordingly, it is necessary to transform the host cell, and to enable the control of expression of the RecX-like protein externally. In order to enable the control of expression of the RecX-like protein, for example, as described above, there is considered a method in which a promoter of the RecX-like protein gene originally contained in the genome of the host cell is modified to an exogenous, controllable promoter. Further, there is also considered a method in which a cassette of an exogenous RecX-like protein gene bearing a controllable promoter is introduced into the genome of the host cell. However, such transformation for the genome of the host cell has technical difficulties in many cases. Also, much time and efforts are needed in many cases. In contrast, if the RecX-like protein gene is inserted into an expression vector which can be amplified in the host cell and this vector is introduced into the host cell, it is possible to constitute easily a host cell which can control the RecX-like protein.

Furthermore, in any of the above-mentioned nucleic acid recombination methods, the method is preferably a nucleic acid recombination method, wherein the expression vector has an inducible promoter which induces the RecX-like protein gene, and at the RecA inhibition step, the RecX-like protein gene is induced by inducing the host cell to express the RecX-like protein.

According to the present invention, the above-mentioned expression vector has an inducible promoter which induces the RecX-like protein gene. With such expression vector, it is possible to express the RecX-like protein from the RecX-like protein gene by inducing the host cell. Therefore, at the RecA inhibition step, the RecX-like protein can be easily expressed by simply inducing the host cell.

The “inducible promoter” is not limited as long as it can express a RecX-like protein by inducing the host cell. The inducible promoter includes an IPTG inducible promoter induced by IPTG, a saccharide inducible promoter induced by saccharide, an alcohol inducible promoter induced by alcohol, a heat inducible promoter induced by heating, and the like.

Furthermore, in any of the above-mentioned nucleic acid recombination methods, the method is preferably a nucleic acid recombination method, wherein at the nucleic acid recombination step, a target nucleic acid containing a target region for recombination, and a recombination vector which comprises a homologous recombination region capable of homologous recombination with the target region, are introduced into the host cell, and the target region is recombined into the recombination vector.

As described above, the nucleic acid recombination method of the present invention can be also carried out between an exogenous target nucleic acid and a genome of the host cell, or between different regions in the genome of the host cell. However, when the target region is desired to be cloned by using this nucleic acid recombination method, there are many cases in which the analysis after cloning, for example, the analysis for sequence and expression becomes difficult if the target nucleic acid is incorporated into the genome.

In contrast, in the present invention, a target nucleic acid containing a target region for recombination, and a recombination vector which comprises a homologous recombination region capable of homologous recombination with the target region, are introduced into the host cell, and the target region is recombined into the recombination vector. In this way, it is possible to easily extract the recombinant (the recombination vector for which recombination is carried out), which can be easily used for the subsequent analysis. Further, there is also an advantage in that the formation of the homologous recombination region in the vector is easier than the formation of the homologous recombination region in a genome.

Here, the “target nucleic acid” is not specifically limited. In other words, any nucleic acid composed of any base sequence may be used, and the chain length is not limited by any upper limit. Accordingly, for example, even a giant DNA having a full length of 3000 Mbp of the human genome may be used. It is needless to say that the origin thereof is not limited. Accordingly, it includes a DNA derived from the genome of a virus or a microorganism, or an animal or a plant, or a modified DNA thereof, or a plasmid, etc. contained in microorganisms, etc., or a chimera DNA formed by insertion of a heterogeneous DNA fragment into the plasmid, etc., or an artificially synthesized oligonucleotide, etc. Further, a cDNA may be also used.

The “homologous recombination region” is not limited if it has a pair of homologous regions having high homology to the regions of the both ends of a target region for cloning. Accordingly, the homologous recombination region may have only a pair of homologous regions, or a pair of homologous regions containing other region between them. It is only necessary if a pair of homologous regions may be substantially homologous to the extent such that a substantial number of the sequences are capable of recombination with the regions respectively at the both ends of the target region. However, since it is possible to carry out the recombination more securely as the homology is higher, the homologous region preferably has homology as high as possible, and most preferably 100% homology.

Furthermore, in any of the above-mentioned nucleic acid recombination methods, the method is preferably a nucleic acid recombination method, wherein an expression vector is composed of a pBR-based vector, and at the nucleic acid recombination step, a target nucleic acid containing a target region for recombination, and a recombination vector which comprises a homologous recombination region capable of homologous recombination with the target region and is composed of a pUC-based vector, are introduced into a host cell, and the target region is recombined into the recombination vector.

According to the present invention, the expression vector is composed of a pBR-based vector, and the recombination vector is composed of a pUC-based vector. Since the pBR-based vector and the pUC-based vector are very easy for handling as compared with other vectors such as a phage, a cosmid etc., these vectors may be conveniently used as the expression vector and the recombination vector. However, if both of the expression vector and the recombination vector are the pBR-based vector or the pUC-based vector, the expression vector and the recombination vector may not coexist sometimes in a host cell. Accordingly, it is preferable that the expression vector is the pBR-based vector and the recombination vector is the pUC-based vector, or the expression vector is the pUC-based vector and the recombination vector is the pBR-based vector. Also, in general, the number of copies of the pUC-based vector in the host cell is larger than that of the pBR-based vector. Since the recombination vector is usually extracted for analysis after the homologous recombination, it is preferably one having the number of the copies as large as possible for ensuring the yield. Accordingly, as in the present invention, it is preferable to use the pUC-based vector as the recombination vector and the pBR-based vector as the expression vector.

Furthermore, in any of the above-mentioned nucleic acid recombination methods, the method is preferably a nucleic acid recombination method, wherein an incubation step to incubate the host cell is provided after the RecA inhibition step.

Since the host cell can be proliferated in a large amount by conducting the incubation step in this way, this is convenient for carrying out analysis such as an analysis for sequence and expression of a recombined region (for example, a cloned target region).

Furthermore, in the nucleic acid recombination method described above, the method is preferably a nucleic acid recombination method which comprises a first incubation step, wherein the expression vector has a first toxic substance resistant gene which gives resistance against the first toxic substance to the host cell, and the incubation step is carried out in the presence of the first toxic substance.

The expression vector introduced into a host cell may sometimes drop out of the host cell in the course of carrying out incubation. In contrast, in the present invention the expression vector has a first toxic substance resistant gene which gives resistance against the first toxic substance to the host cell. Further, the present invention comprises the first incubation step to carry out incubation in the presence of the first toxic substance. If incubation is carried out in the presence of the first toxic substance in this way, since the host cell cannot be viable or cannot proliferate in the absence of the expression vector, the expression vector is made to be present always in the host cell. Accordingly, the dropout of the expression vector can be prevented.

Here, the “first toxic substance” is not limited as long as it can destroy or cease the growth of a host cell without an expression vector. For example, an antibiotic such as streptomycin, kanamycin, tetracycline, penicillin, cephalosporin, erythromycin, leukomycin, etc. may be used.

Further, the “first toxic substance resistant gene” may be any gene as long as it can give resistance against the first toxic substance the host cell.

In addition, for the incubation step, the first incubation step to carry out incubation in the presence of the first toxic substance, may constitute the whole incubation step, or may be a part of the incubation step. However, to ensure prevention of the dropout of the expression vector, the first incubation step preferably constitutes the whole incubation step.

Furthermore, in any of the above-mentioned nucleic acid recombination methods, the method is preferably a nucleic acid recombination method which comprises a second incubation step, wherein the recombination vector has a second toxic substance resistant gene which gives resistance against the second toxic substance to the host cell, and the incubation step is carried out in the presence of the second toxic substance.

Also, the recombination vector introduced into a host cell may sometimes drop out of the host cell in the course of carrying out incubation. In contrast, in the present invention the recombination vector has the second toxic substance resistant gene which gives resistance against the second toxic substance to the host cell. Further, the present invention comprises the second incubation step to carry out incubation in the presence of the second toxic substance. If incubation is carried out in the presence of the second toxic substance in this way, since the host cell cannot be viable or cannot proliferate in the absence of the recombination vector, the recombination vector is made to be present always in the host cell. Accordingly, the dropout of the recombination vector can be prevented.

Here, the “second toxic substance” is not limited as long as it can destroy or cease the growth of a host cell without a recombination vector, as in the case of the first toxic substance.

Further, the “second toxic substance resistant gene” may be any gene as long as it can give resistance against the second toxic substance to the host cell.

However, if the first toxic substance is the same as the second toxic substance when the host cell contains both of the expression vector and the recombination vector, since the host cell can be viable and can proliferate in the presence of either one of the vectors, the other vector drops out of the host cell. Accordingly, when the host cell comprises both of the expression vector and the recombination vector, the first toxic substance and the second toxic substance are preferably different from each other.

Further, for the incubation step, the second incubation step to carry out incubation in the presence of the second toxic substance may constitute the whole incubation step, or may be a part of the incubation step. However, to ensure prevention of the dropout of the recombination vector, the second incubation step preferably constitutes the whole incubation step. Also, when there is a first incubation step, the first incubation step and the second incubation step can be carried out at the same time, i.e., incubation can be carried out in the presence of both of the first toxic substance and the second toxic substance. By performing the incubation steps at the same time, the number of the working steps can be decreased, and the dropout of either one of the vectors during the incubation can be prevented.

Furthermore, in any of the above-mentioned nucleic acid recombination methods, the method is preferably a nucleic acid recombination method, wherein the incubation step comprises a plate incubation step to incubate the host cell on a plate medium to form a plurality of colonies on the plate medium, and a single colony incubation step to incubate separately each colony collected from the plate medium; and
the nucleic acid recombination method is provided with a nucleic acid extraction step to extract the nucleic acid which contains at least the recombinant vector from the host cell incubated by the incubation step using the single colony incubation step, and an electrophoresis step to electrophorese the extracted nucleic acid.

According to the present invention, the incubation step comprises a plate incubation step to incubate the host cell on a plate medium to form a plurality of colonies on the plate medium, and a single colony incubation step to incubate separately each colony collected from the plate medium. By conducting such incubation, the host cell can be easily separated. Further, the present invention comprises a nucleic acid extraction step to extract the nucleic acid which contains at least the recombinant vector from the host cell incubated by the incubation step using the single colony incubation step, and an electrophoresis step to electrophorese the extracted nucleic acid. Accordingly, it can be easily checked whether or not desired recombination has been done in the separated host cell. For example, it can be easily checked whether or not desired cloning has occurred.

In addition, when the first incubation step and the second incubation step are included, the plate incubation step is preferably carried out at the same time with these steps. Also when the first incubation step and the second incubation step are included, the single colony incubation step is preferably carried out at the same time with these steps. In this way, it is possible to prevent the dropout of the expression vector or the recombination vector during the plate incubation step or the single colony incubation step.

Further, another means for solving such problems is a host cell which enables homologous recombination of nucleic acids in its own cell, and is constituted to be able to express a RecA-like protein with ability of recombination and to express a RecX-like protein.

The host cell of the present invention is constituted to be able to express the RecA-like protein with ability of recombination and to express the RecX-like protein.

With such host cell, since it is possible to use the RecA-like protein when it is needed and to use the RecX-like protein to inhibit the activity of the RecA-like protein when it is not needed, it is possible to carry out recombination between desired DNA regions only while suppressing recombination between undesired DNA regions. Accordingly, it is possible to carry out precisely homologous recombination of a desired target region in the host cell. Moreover, it is also possible to improve recombination efficiency as compared with that of the conventional methods although the reason is not clearly understood.

Therefore, for example, if such host cell is used in cloning, it is possible to clone the target region for cloning in its intact state into a vector. It is needless to say that, with such method, it is also possible to solve the problems of the method disclosed in Non-Patent Document 1, i.e., the problem in that the target region cannot be cloned as a whole, etc., and the problems of the method disclosed in Non-Patent Document 2, i.e., the problem in that mutation may occur at the target region, etc.

The “host cell”, the “RecA-like protein”, the “RecX-like protein”, etc. are the same as in the description of the nucleic acid recombination method.

Further, the RecA-like protein may be expressed at least at the time of homologous recombination of nucleic acids, i.e., it may be expressed constantly in a host cell, or, it may be expressed at the time of recombination, even if not constantly. For example, if the host cell originally expresses the RecA-like protein constantly, the gene of the host cell may be used as it is for the RecA-like protein gene. Further, a suitable inducible promoter may be attached to the RecA-like protein gene. In this way, it is possible to induce the RecA-like protein gene by inducing the host cell to express the RecA-like protein.

Further, the RecX-like protein is desirably expressed when activity of the RecA-like protein is desired to be inhibited. For example, a suitable inducible promoter is desirably attached to the RecX-like protein gene. In this way, it is possible to induce the RecX-like protein gene by inducing the host cell to express the RecX-like protein.

Furthermore, in the above-mentioned host cell, the host cell is preferably a host cell wherein the above-mentioned RecA-like protein is the RecA protein of Escherichia coli.

As described above, the RecA-like protein is not limited as long as it has a function similar to that of the RecA protein of E. coli in homologous recombination. However, the RecA-like protein used is most preferably the RecA protein of E. coli from the viewpoint that the RecA protein gene which encodes the RecA protein of E. coli is easily available and has been most studied for its functions and properties.

Furthermore, in any of the above-mentioned host cells, the host cell is preferably a host cell wherein the above-mentioned the RecX-like protein is the RecX protein of Escherichia coli.

As described above, the RecX-like protein is not limited as long as it has a function similar to that of the RecX protein of E. coli from the viewpoint that it inhibits the activity of the RecA-like protein. However, the RecX-like protein used is most preferably the RecX protein of E. coli from the viewpoint that the RecX protein gene which encodes the RecX protein of E. coli is easily available, and that RecX-like proteins other than the RecX protein of E. coli have not been studied in detail for their functions and properties.

Furthermore, in any of the above-mentioned host cells, the host cell is preferably Escherichia coli.

As described above, the host cell is not limited as long as it is constituted to be able to express the RecA-like protein with ability of recombination and to express the RecX-like protein. However, the host cell is most preferably E. coli considering easy availability and easy handling. In particular, when cloning is performed using the host cell, it is preferable to use E. coli from a viewpoint of cloning efficiency and ease of subsequent analysis.

Furthermore, in any of the above-mentioned host cells, the host cell is preferably a host cell prepared from a cell having a RecA-like protein gene expressing the above-mentioned RecA-like protein in its original genome.

As described above, the RecA-like protein may not necessarily be expressed constantly as long as it is expressed at least at the time of recombination. Since the RecA-like protein gene existing originally in the genome of the host cell usually expresses the RecA-like protein constantly, such RecA-like protein gene can be used as it is. As described above, if the RecA-like protein gene contained in its original genome is used, labors for constructing the host cell may be spared since there is no particular need to transform the host cell for the RecA-like protein gene.

Furthermore, in any of the above-mentioned host cells, the host cell is preferably a host cell which has an expression vector into which a RecX-like protein gene expressing the RecX-like protein is inserted.

As described above, the RecX-like protein should be controlled to be expressed when the activity of the RecA-like protein is desired to be inhibited. On the other hand, the RecX-like protein gene contained originally in the host cell is in general not controllable externally for its expression. Accordingly, it is necessary to transform the host cell, and to enable the control of expression of the RecX-like protein externally. In order to enable the control of expression of the RecX-like protein, for example, as described above, there is considered a method in which a promoter of the RecX-like protein gene originally contained in the genome of the host cell is modified to an exogenous, controllable promoter. Further, there is also considered a method in which a cassette of an exogenous RecX-like protein gene bearing a controllable promoter is introduced into the genome of the host cell. However, such transformation for the genome of the host cell has technical difficulties in many cases. Also, much time and efforts are needed in many cases. In contrast, if the RecX-like protein gene is inserted into the expression vector which can be amplified in the host cell and this vector is introduced into the host cell, it is possible to constitute easily the host cell which can control the RecX-like protein.

Furthermore, in the above-mentioned host cell, the host cell is preferably a host cell wherein the expression vector has an inducible promoter which induces the RecX-like protein gene, and the RecX-like protein gene is induced by inducing the host cell and the RecX-like protein is expressed.

According to the present invention, the above-mentioned expression vector has an inducible promoter which induces the RecX-like protein gene. With such expression vector, it is possible to express the RecX-like protein from the RecX-like protein gene by inducing the host cell. Therefore, when the activity of the RecA-like protein is desired to be inhibited, the RecX-like protein can be easily expressed by simply inducing the host cell.

The “inducible promoter” is as explained in the description of the nucleic acid recombination method.

Furthermore, in any of the above-mentioned host cells, the host cell is preferably a host cell wherein the expression vector is a pBR-based vector.

According to the present invention, the expression vector is composed of the pBR-based vector. Since the pBR-based vector is very easy to handle compared to other vectors such as a phage vector and a cosmid vector, this vector may be conveniently used as the expression vector. Further, when recombination is carried out by introducing a recombination vector into the host cell, the expression vector and the recombination vector can easily coexist in the host cell by using a pUC-based vector as the recombination vector. Also, the number of copies of the pUC-based vector is generally larger than that of the pBR-based vector in the host cell. Since the recombination vector is usually extracted as a recombinant for analysis following the homologous recombination, it is preferably one having the number of the copies as large as possible for ensuring the yield. Accordingly, it is preferable to use the pBR-based vector as the expression vector and the pUC-based vector as the recombination vector.

Furthermore, in any of the above-mentioned host cells, the host cell is preferably a host cell wherein the expression vector has a first toxic substance resistant gene which gives resistance against the first toxic substance to the host cell.

The expression vector introduced into a host cell may sometimes drop out of the host cell in the course of carrying out incubation. In contrast, in the present invention the expression vector has a first toxic substance resistant gene which gives resistance against the first toxic substance to the host cell. For this reason, if incubation is carried out in the presence of the first toxic substance, since the host cell cannot be viable or cannot proliferate in the absence of the expression vector, the expression vector is made to be present always in the host cell. Accordingly, the dropout of the expression vector can be prevented.

The “first toxic substance” and the “first toxic substance resistant gene” are as explained in the description of the nucleic acid recombination method.

Further, another means for solving such problems is an expression vector which is introduced into a host cell and expresses a protein in the host cell, and has a RecX-like protein gene expressing a RecX-like protein.

If such expression vector is introduced into a host cell which is capable of expressing a RecA-like protein with ability of recombination, it is possible to use the RecA-like protein when the RecA-like protein is needed, and it is possible to inhibit the activity of the RecA-like protein by the RecX-like protein when the RecA-like protein is not needed. Therefore, in the host cell into which the expression vector is introduced, it is possible to carry out recombination between desired nucleic acid regions only and to suppress the recombination between undesired nucleic acid regions. Accordingly, it is possible to carry out precisely homologous recombination of a desired target region in the host cell. Furthermore, it is possible to improve the efficiency of recombination compared to previous cases. Therefore, if the host cell into which such expression vector is introduced is used in cloning, it is possible to clone the target region for cloning in its intact state into the vector.

The “host cell”, the “RecX-like protein”, the “RecX-like protein gene”, etc. are as explained in the description of the nucleic acid recombination method.

Furthermore, in the above-mentioned expression vector, the expression vector is preferably an expression vector which has an inducible promoter which induces the RecX-like protein gene and expresses the RecX-like protein.

According to the present invention, the expression vector has an inducible promoter which induces the RecX-like protein gene. If such expression vector is introduced into a host cell, it is possible to express the RecX-like protein from the RecX-like protein gene by inducing the host cell. Therefore, when the activity of the RecA-like protein is desired to be inhibited, the RecX-like protein can be easily expressed by simply inducing the host cell.

The “inducible promoter” is as explained in the description of the nucleic acid recombination method.

### BRIEF DESCRIPTION OF THE DRAWINGS

FIG. 1 is an explanatory diagram showing the structure of an expression vector having an inserted RecX protein gene in reference to Example 1.

FIG. 2 is an explanatory diagram showing the structure of a target nucleic acid which contains a target region for cloning in reference to Example 1.

FIG. 3 is an explanatory diagram showing the structure of a recombination vector having an inserted homologous recombination region to incorporate a target region in reference to Example 1.

FIG. 4 is a photograph instead of a drawing showing the results of the electrophoresis for the extracted plasmid DNA in reference to Example 1.

FIG. 5 is a photograph instead of a drawing showing the results of the electrophoresis for the extracted plasmid DNA in reference to Example 2.

FIG. 6 is a photograph instead of a drawing showing the results of the electrophoresis for the extracted plasmid DNA in reference to Example 3.

FIG. 7 is a photograph instead of a drawing showing the results of SDS-PAGE for the protein extracted from the host cell in reference to Example 4.

FIG. 8 is an explanatory diagram showing a method of cloning a target DNA by using a restriction enzyme in reference to a conventional technique.

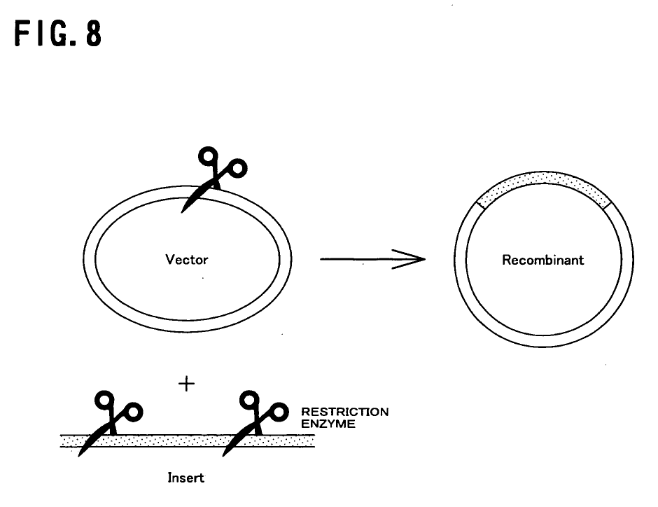

FIG. 9 is an explanatory diagram showing a method of cloning a target DNA by using a PCR reaction in reference to a conventional technique.

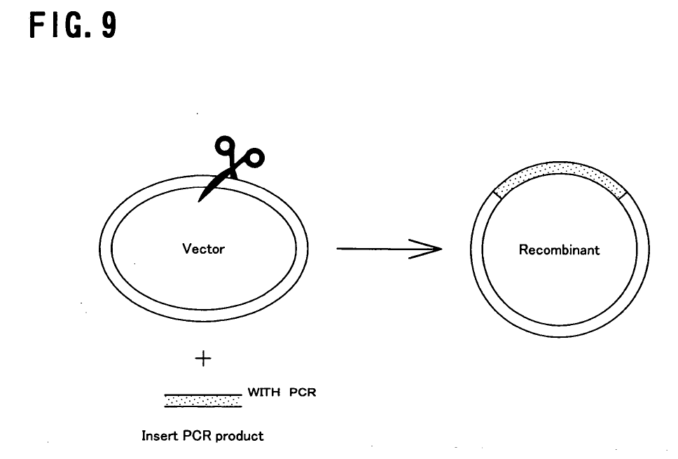

FIG. 10 is an explanatory diagram showing a method of cloning a target DNA by using homologous recombination of a nucleic acid in reference to a conventional technique.

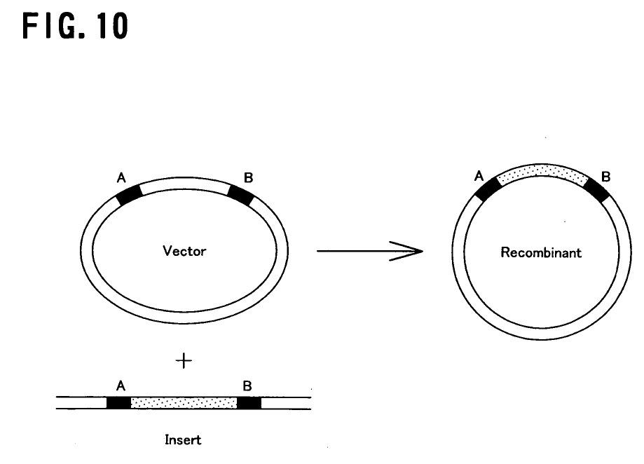

### DETAILED DESCRIPTION

### EXAMPLE 1

Examples of the present invention will be further illustrated below with reference to Drawings.

First, an Escherichia coli JC8679 strain (a kit manufactured by Invitrogen Corporation) was provided to construct a host cell which enables homologous recombination of a nucleic acid in its own cell, and the strain was modified to a DE3 form according to the protocol. This E. coli JC8679 strain is a wild strain having originally a RecA protein gene expressing a RecA protein in its own genome. And, this E. coli JC8679 strain expresses the RecA protein constantly, and is capable of homologous recombination.

In addition, for the base sequence of the RecA protein gene of E. coli, refer to GenBank ACCESSION No.: V00328 J01672.

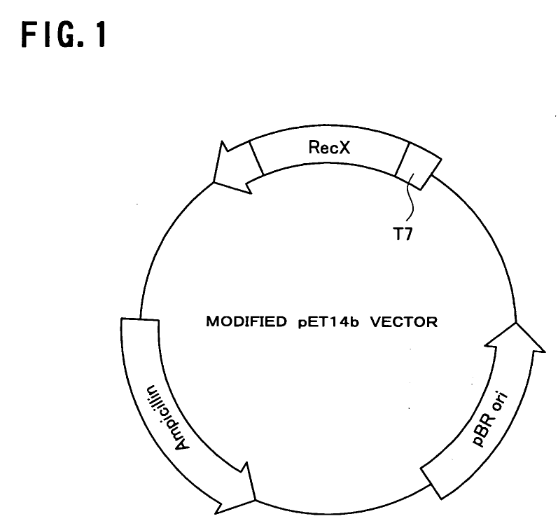

On the other hand, as shown in FIG. 1, a modified pET14b vector was prepared by modification of a pET14 vector (NOVAGEN) comprising a pBR-based plasmid vector as an expression vector which is introduced into a host cell to express a protein in the host cell. This modified pET14b vector is a vector prepared by inserting the RecX protein gene of Escherichia coli into the pET14b vector. Specifically, this modified pET14b vector has an ampicillin resistant gene (first toxic substance resistant gene) which gives ampicillin (the first toxic substance) resistance to the host cell, i.e., E. coli JC8679 strain. This modified pET14b vector also has an IPTG inducible promoter (T7) which is induced by IPTG. Further, this modified pET14b vector has the RecX protein gene of Escherichia coli which expresses the RecX protein downstream of the IPTG inducible promoter.

This expression vector can be prepared by a method known in the art. Further, for the base sequence of the RecX protein gene of E. coli, refer to De Mot, R., Schoofs, G. & Vanderleyden, J. (1994) Nucleic Acids Res. 22, 1313-1314, and Vierling, S., Weber, T., Wohlleben, W. & Muth, G. (2000) J. Bacteriol. 182, 4005-4011.

Next, to the DE3-modified E. coli JC8679 strain (hereinafter, also referred to as E. coli JC8679 (DE3)), the above-mentioned expression vector was introduced, and a host cell was prepared to carry out homologous recombination of a nucleic acid. Such host cell is constituted to be able to express a RecA protein constantly with ability of recombination and to express a RecX protein externally. In other words, this host cell expresses the RecA protein constantly, and homologous recombination of the nucleic acid can be carried out in the cell. Further, by inducing the host cell with IPTG, it is possible to induce the RecX protein gene on the expression vector to express the RecX protein, and consequently to inhibit the activity of the RecA protein.

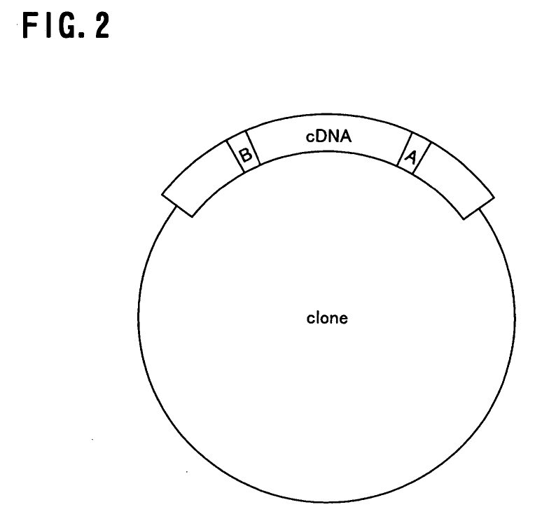

Further, as shown in FIG. 2, a cDNA clone was prepared as a target nucleic acid which contains a target region for recombination. Such target nucleic acid (cDNA clone) is one of cDNA clones made from a human brain cell. The vector was pBluescript II SK+ (Toyobo Co., Ltd.). For the base sequence of the inserted fragment (cDNA) of the clone, refer to a part corresponding to Exon 11 of Human p53 gene for transformation related protein p53 GenBank ACCESSION No.: X54156. In addition, the sequences of both ends of the target region for cloning (Sequence A and Sequence B) are regions for homologous recombination in the following recombination reaction.

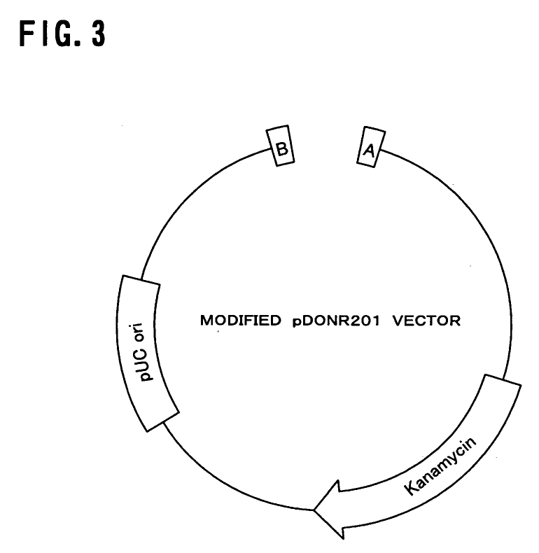

Further, on the other hand, as shown in FIG. 3, a modified pDONR201 vector was prepared by modification of a pDONR201 vector (manufactured by Invirtogen Corporation) consisting of a pUC-based vector as a recombination vector to incorporate the target region. In this modified pDONR201 vector, homologous sequences (Homologous sequence A and Homologous sequence B) to the sequences of both ends of the target region among the target nucleic acid (cDNA clone) (Sequence A and Sequence B) are inserted into the predetermined locations of the pDONR201 vector. Specifically, this modified pDONR201 vector is formed by a PCR reaction with a pair of primer DNAs shown below (Oligonucleotide 1 and Oligonucleotide 2) on the base of pDONR201 vector. Accordingly, this modified pDONR201 vector is already linearized. Homologous region A and Homologous region B are sequences 100% homologous, respectively, to Region A and Region B of the target regions of the cDNA clone. In this Example, these Homologous region A and Homologous region B are homologous recombination regions that can homologously recombine with the target regions.

Further, a PCR reaction for preparing the modified pDONR201 vector was conducted in the following manner: at 94° C. for 5 minutes, 5 cycles (at 94° C. for 30 seconds and at 40° C. for 30 seconds), 20 cycles (at 72° C. for 2 minutes, at 94° C. for 30 seconds and at 55° C. for 30 seconds) and at 72° C. for 2 minutes, followed by keeping at 4° C. In this Example, such a two-stage PCR reaction was conducted since the sequences which can anneal to the pDONR201 vector among the primers are short (specifically, 5′-tttagcttccttagc-3′ for the forward side, and 5′-atgtcaggctccctt-3′ for the reverse side are sequences which can be annealed to the vector).

This modified pDONR201 vector has a kanamycin resistant gene (the second toxic substance resistant gene) which gives kanamycin (the second toxic substance) resistance to the host cell, i.e., E. coli JC8679 strain.

Homologous Region A (Sequence 1):

5′-acaataaaactttgctgcca-3′

Homologous Region B (Sequence 2):

5′-ccttttttgggacttcaggtgg-3′

Oligonucleotide 1 (Sequence 3):

5′-acaataaaactttgctgccatttagcttccttagc-3′

Oligonucleotide 2 (Sequence 4):

5═-cctttttggacttcaggtggatgtcaggctccctt-3′

Next, the above-mentioned E. coli JC8679 (DE3) was modified to competent cells. Then, the competent cells were dissolved in 30 μl of a dissolution solution, mixed with 1 μl of the target nucleic acid (cDNA clone) and 1 μl of the recombination vector (a modified pDONR201 vector), and the resultant solution was immediately kept at 0° C. for 20 minutes. Then, electrophoresis was conducted, followed by keeping at 0° C. for 2 minutes.

In this Example, these procedures correspond to the recombination step of the nucleic acid. Accordingly, at this step, by the RecA protein expressed in E. coli JC8679 (DE3), homologous recombination of the nucleic acid was conducted; specifically, recombination between the target region of the target nucleic acid (cDNA clone) and the homologous recombination region (Homologous region A and Homologous region B) of the recombination vector (modified pDONR201 vector) was conducted. As a result, the target region was incorporated into the recombination vector to form a recombinant plasmid DNA.

Next, the RecA inhibition step and the incubation step was conducted. First, 300 μl of SOC medium containing IPTG was added, and shaken for 1 hour at 37° C., and pre-incubation of the transformed E. coli JC8679 (DE3) was conducted.

In this Example, this procedure corresponds to the RecA inhibition step and a part of the incubation step. Accordingly, at this step, IPTG induced E. coli JC8679 (DE3), which induced the RecX protein gene, and the RecX protein was expressed to consequently inhibit the activity of the RecA protein.

Next, the pre-incubated E. coli suspension was plated on an LB-Amp, Km plate medium (LB plate medium containing ampicillin and kanamycin) in a suitable amount, and the resultant medium was incubated for 20 hours at 37° C. That is, in this Example, the following steps were conducted at the same time: the first incubation step of carrying out incubation in the presence of the first toxic substance (kanamycin), the second incubation step of carrying out incubation in the presence of the second toxic substance (ampicillin), and the plate incubation step of incubating the host cell on a plate medium to form a plurality of colonies on the plate medium. After the incubation, the medium was kept at 4° C. for about 3 days.

Then, each colony of the transformed strain collected from the plate medium was incubated separately for 20 hours on an LB-Amp, Km liquid medium (LB liquid medium containing ampicillin and kanamycin). That is, in this Example, the following steps were conducted at the same time: the first incubation step of carrying out incubation in the presence of the first toxic substance (kanamycin), the second incubation step of carrying out incubation in the presence of the second toxic substance (ampicillin), and the single colony incubation step of incubating the single colony.

Next, the plasmid DNA was isolated and purified from this incubation solution (nucleic acid extraction step). Further, isolation and purification of the plasmid DNA may be carried out by a method known in the art.

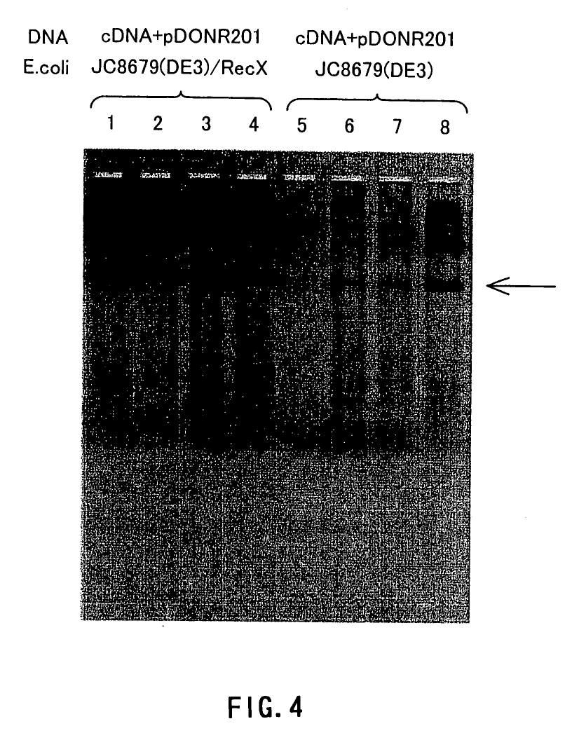

Then, the extracted plasmid DNA was subjected to electrophoresis with 1.2% agarose gel (electrophoresis step). The gel was stained with Et-Br and was recorded on a photograph with UV irradiation. The results are shown in FIG. 4.

Lane 1 to Lane 4 are the results obtained by conducting the above-described reaction, which are the results obtained by performing electrophoresis of the plasmid DNA isolated and purified from each of the different E. coli colonies.

Lane 5 to Lane 8 are the results for the Comparative Example, in which E. coli JC8679 strain was used as it is as the host cell for transformation. That is, E coli having the RecA protein gene but not an expression vector (modified pET14b vector) which contains the RecX protein gene was used. Other procedures except this were the same as those in Lane 1 to Lane 4. Lane 5 to Lane 8 are also the results of electrophoresis of the plasmid DNA isolated and purified from each of the different E. coli colonies.

The signal seen at the position indicated by the arrow in FIG. 4 is the recombinant plasmid DNA in which the target region of the target nucleic acid (cDNA clone) was incorporated into the recombination vector (modified pDONR201 vector).

As is clear from the results of FIG. 4, in any of Lane 1 to Lane 4, which are the results for the Example, the recombinant plasmid DNA was obtained stably. Further, as far as it is shown in the results of electrophoresis, it can be inferred that undesired recombination does not occur.

The plurality of signals seen above the signal at the position indicated by the arrow are considered to be multimers such as dimers and trimers of the recombinant plasmid DNA. The reasoning is based on the following: when these DNAs are isolated, extracted, cleaved by a suitable restriction enzyme, and identified with electrophoresis, any one derived from any signal is detected as a signal of the same size. Also, from the results of Southern hybridization with a suitable probe, the ones treated with the restriction enzyme show the same results for any one derived from any signal.

On the other hand, in Lane 5 to Lane 8 which are the results for the Comparative Example, the recombinant plasmid DNA was not obtained, or was remarkably reduced in its amount in many cases (see Lane 5 to Lane 7), and unstable. Further, the plurality of signals seen above the signal at the position indicated by the arrow are considered to be multimers of the recombinant plasmid DNA, similarly to those of Lane 1 to Lane 4 of the Example.

From these results, it is found that the recombinant plasmid DNA is obtained stably by expressing the RecX protein. It is also found that undesired recombination can be suppressed by expressing the RecX protein. On the other hand, it is found that the recombinant plasmid DNA is not obtained stably when the RecX protein is not expressed.

Further, the reason for which the recombinant plasmid DNA is not obtained stably in the Comparative Example, is considered to be that the recombinant plasmid DNA has dropped out of the host cell due to the long standing time (about 3 days) after the plate incubation (i.e., since the recombinant plasmid DNA was present for a too long time in the host cell). As in Examples 2 and 3 described below, if the standing time after the plate incubation is shortened (if the time for which plasmid DNA is present in the host cell is shortened), the recombinant plasmid DNA can be obtained relatively stably. However, when the plasmid is extracted from E. coli from a plurality of samples by using an automatic plasmid isolation apparatus, the time for which the plasmid DNA is present in the host cell becomes long due to the operating conditions of the apparatus. Consequently, the recombinant plasmid DNA cannot be obtained sufficiently as seen in the Comparative Example.

On the other hand, in the Example, even if the recombinant plasmid DNA is present for a long time in the host cell by, for example, extending the incubation time, standing for a long time after the plate incubation, etc., there is no dropout of the recombinant plasmid DNA. Accordingly, even when the plasmid is extracted from E. coli from the plurality of samples by using the automatic plasmid isolation apparatus, the recombinant plasmid DNA can be obtained stably.

Furthermore, when the number of colonies is compared after carrying out the plate incubation step between the Example and the Comparative Example, it was found that the results obtained in the Example was about 20 times as much as the results obtained in the Comparative Example in spite of the same conditions. From these results, it is also found that the recombinant plasmid DNA is obtained stably by expressing the RecX protein. It is found that the transformation efficiency (recombination efficiency) is increased greatly as compared with those known in the art.

As explained above, the nucleic acid recombination method (cloning method) of the present Example comprises a recombination step of a nucleic acid to carry out homologous recombination of the nucleic acid by a RecA protein expressed in E. coli JC8679 (DE3), and a RecA inhibition step to inhibit the activity of the RecA protein by expressing the RecX protein. In such method, when the RecA-like protein is needed, the RecA-like protein can be used, and when the RecA-like protein is not needed, its activity can be inhibited by the RecX-like protein. Thus it is possible to carry out recombination between desired nucleic acid regions only by suppressing the recombination between undesired nucleic acid regions. Accordingly, it is possible to carry out precisely homologous recombination of a desired target region. Further, the recombination efficiency can be increased as compared to a prior art. Therefore, it is possible to clone the target region for cloning in its intact state into the recombination vector (modified pDONR201 vector). Further, the problems of techniques known in the art are also solved.

Further, in this Example, the RecA protein of E. coli was used as the RecA-like protein. The RecA protein of E. coli is considered to be most preferable from the viewpoint that the RecA protein of E. coli has been most studied for its functions and properties.

Further, in this Example, the RecX protein of E. coli was used as the RecX-like protein. The RecX protein of E. coli is considered to be most preferable from the viewpoint that the RecX protein gene which encodes the RecX protein of E. coli is easily available, and that RecX-like proteins other than the RecX protein of E. coli have not been studied in detail for their functions and properties.

Further, in this Example, E. coli was used as the host cell. E. coli is most preferable considering easy availability and easy handling. Especially, when cloning is conducted by using the nucleic acid recombination method as in the present Example, E. coli is suitable in view of cloning efficiency and ease of subsequent analysis.

Further, in this Example, the host cell was prepared from E. coli JC8679 (DE3) having originally the RecA protein gene expressing the RecA protein in its own genome. Since the RecA protein gene existing originally in the genome of E. coli JC8679 (DE3) expresses the RecA protein constantly, such RecA protein gene can be used as it is. In this way, if the RecA protein gene which is contained originally in its own genome is used, it is not necessary to specially transform E. coli JC8679 (DE3) with respect to the RecA protein gene, and therefore, labors for constructing the host cell can be spared.

Further, in this Example, the host cell has an expression vector (modified pET14b vector) into which the RecX protein gene is inserted, and subjects the RecX protein gene to express the RecX protein. In this way, it is possible to constitute comparatively easily the host cell which can control the RecX protein.

Further, in this Example, the expression vector (modified pET14b vector) has the inducible promoter (IPTG inducible promoter) which induces the RecX protein gene. With such expression vector, it is possible to express the RecX protein from the RecX protein gene by inducing the host cell. Therefore, at the RecA inhibition step, the RecX protein can be easily expressed by simply inducing the host cell.

Further, in this Example, together with the target nucleic acid (cDNA clone) which contains the target region, the recombination vector (modified pDONR201 vector) which contains the homologous recombination region (Homologous region A and Homologous region B) that can homologously recombine with the target region is introduced into the host cell, thereby the target region is recombined into the recombination vector. In this way, it is possible to easily extract the recombinant plasmid DNA, which is useful for subsequent analysis. Further, the formation of the homologous recombination region in the recombination vector is easier than the formation of the homologous recombination region in the genome of E. coli.

Further, in this Example, the expression vector is the pBR-based vector and the recombination vector is the pUC-based vector. Since the pBR-based vector and the pUC-based vector are very easy for handling, these vectors can be conveniently used as the expression vector and the recombination vector. Further, in this way, the expression vector and the recombination vector can coexist in E. coli. Also, since the number of copies of the pUC-based vector in the cell is generally larger than that of the pBR-based vector, use of the pUC-based vector as the recombination vector makes it possible to easily ensure the yield of the recombinant DNA in the nucleic acid extraction step.

Further, in this Example, the incubation step to incubate the host cell was provided after the RecA inhibition step. By performing the incubation step in this way, since the host cell can be proliferated in a large amount, it is convenient for carrying out analysis etc. of the cloned target region.

Furthermore, in this Example, the expression vector (modified pET14b vector) has the ampicillin resistant gene which gives ampicillin resistance to E. coli. And, the first incubation step to carry out incubation in the presence of ampicillin is provided. In this way, since E. coli cannot be viable or cannot proliferate when the expression vector drops out of the E. coli, the expression vector is made to exist always in E. coli. Accordingly, the dropout of the expression vector can be prevented.

Furthermore, in this Example, the recombination vector (modified pDONR201 vector) has the kanamycin resistant gene which gives the kanamycin resistance to E. coli. And, the second incubation step to carry out incubation in the presence of kanamycin is provided. In this way, since E. coli cannot be viable or cannot proliferate when the recombination vector drops out of the E. coli, the recombination vector is made to exist always in E. coli. Accordingly, the dropout of the recombination vector can be prevented.

Further, since the first toxic substance and the second toxic substance used are different, the dropout of either the expression vector or the recombination vector can be prevented. Also, since the whole incubation step is composed of the first incubation step and the second incubation step, the dropout of either of the vectors can be prevented.

Furthermore, in this Example, the incubation step comprises the plate incubation step to incubate E. coli on the plate medium to form a plurality of colonies on the plate medium, and the single colony incubation step to incubate separately each colony collected from the plate medium. By conducting such incubation, E. coli can be easily separated. Further, the nucleic acid extraction step to extract the plasmid from the E. coli incubated in the single colony incubation step, and the electrophoresis step to electrophorese the plasmid are provided. Accordingly, it is possible to easily identify whether or not the separated E. coli is the one with the desired recombination. In other words, it is possible to easily identify whether or not the desired cloning has been made.

### EXAMPLE 2

Next, Example 2 will be explained. Explanation for the parts similar to those of Example 1 will be omitted or simplified.

First, Escherichia coli JC8679 strain (DE3) was prepared by introducing a modified pET14b vector, to express a RecX protein in the same manner as in Example 1. As described above, the E. coli JC8679 (DE3) is constituted to enable the expression and recombination of a RecA protein, and the expression of the RecX protein by induction with IPTG.

Further, a cDNA clone was prepared in the same manner as in Example 1. Further, a modified pDONR201 vector was prepared as a recombination vector to incorporate a target region in the same manner as in Example 1.

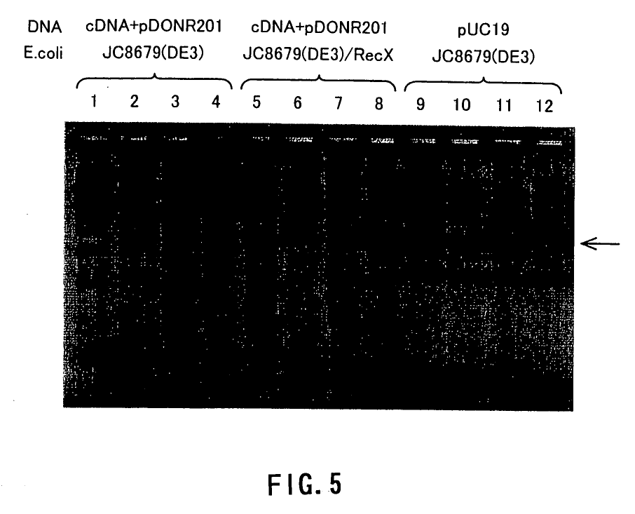

Next, transformation and further an incubation step were carried out basically in the same manner as in Example 1. However, the standing time after the plate incubation was set for about 2 days at 4° C. And, a plasmid DNA derived from a single colony was extracted (nucleic acid extraction step), and agarose gel electrophoresis was conducted (electrophoresis step), followed by recording on a photograph in the same manner as in Example 1. The results are shown in FIG. 5.

Lane 5 to Lane 8 are the results obtained by conducting the above-described reaction, showing the results of electrophoresis for the plasmid DNA that was isolated and purified from each separate E. coli colony.

Lane 1 to Lane 4 are the results of a Comparative Example, in which E. coli JC8679 strain was used as it is as a host cell for transformation. That is, the E coli JC8679 strain without an expression vector which contains the RecX protein gene was used. Other procedures except this were the same as those in Lane 5 to Lane 8. Lane 1 to Lane 4 are also the results of electrophoresis for the plasmid DNA that was isolated and purified from each separate E. coli colony.

Lane 9 to Lane 12 are also the results of the Comparative Example, in which E. coli JC8679 strain was used as it is as the host cell similarly to Lane 1 to Lane 4. Further, in these Lanes, the target nucleic acid was not added, and a pUC 19 vector was introduced instead of the above-mentioned recombination vector (modified pDONR201 vector). Other procedures except this were the same as those in Lane 5 to Lane 8.

The signal seen at the position indicated by the arrow in FIG. 5 is the recombinant plasmid DNA in which the target region of the target nucleic acid (cDNA clone) was incorporated into the recombination vector (modified pDONR201 vector).

As is clear from the results of FIG. 5, in any of Lane 5 to Lane 8, which are the results for the Example, the recombinant plasmid DNA was obtained. Further, as far as it is shown in the results of electrophoresis, it can be inferred that undesired recombination does not occur. The plurality of signals seen above the signal at the position indicated by the arrow are considered to be multimers of the recombinant plasmid DNA, similarly to Example 1.

On the other hand, in Lane 1 to Lane 4 which are the results for the Comparative Example, except in Lane 3, the desired recombinant plasmid DNA was not obtained, and a large number of the recombinant plasmid DNAs in which undesired recombination may have occurred was identified.

Further, in Lane 9 to Lane 12 which are the results for the Comparative Example, the vector DNA (pUC 19 vector) only was observed since the target nucleic acid was not added. The signals seen in the figure are considered to be the monomer vector DNA corresponding to the lowest molecular weight and multimers such as dimers and trimers corresponding to higher molecular weight fractions.

From these results, it is found that the recombinant plasmid DNA is obtained stably by expressing the RecX protein. It is also found that undesired recombination can be suppressed by expressing the RecX protein. On the other hand, it is found that when the RecX protein is not expressed, the undesired recombination occurs frequently while the desired recombinant plasmid DNA is not obtained in many cases. Further, it is needless to say that it is found that if there is no target nucleic acid capable of homologous recombination, the recombinant plasmid DNA is not obtained.

Further, in the Comparative Example, if the recombinant plasmid DNA is present for a long time in the host cell by, for example, extending the incubation time after the transformation, extending further the standing time after the plate incubation, etc., the recombinant plasmid DNA drops out of the host cell as shown in the above-mentioned Example 1.

On the other hand, in the Example, even if the recombinant plasmid DNA is present for a long time in the host cell by, for example, extending the incubation time, standing for a long time after the incubation, etc., there is no dropout of the recombinant plasmid DNA, and the recombinant plasmid DNA can be obtained stably while the undesired recombination does not occur.

Further, for the parts similar to those of Example 1, the same effects as those of Example 1 are obtained in this Example.

### EXAMPLE 3

Next, Example 3 will be explained. Explanation for the parts similar to those of Example 1 or 2 will be omitted or simplified.

First, Escherichia coli JC8679 strain (DE3) was prepared by introducing a modified pET14b vector, to express a RecX protein in the same manner as in Examples 1 and 2. The E. coli JC8679 (DE3) is constituted to enable expression and recombination of a RecA protein and expression of the RecX protein by induction with IPTG as described above.

Further, a cDNA clone was prepared in the same manner as in Examples 1 and 2. Further, a modified pDONR201 vector was prepared as a recombination vector to incorporate a target region in the same manner as in Examples 1 and 2.

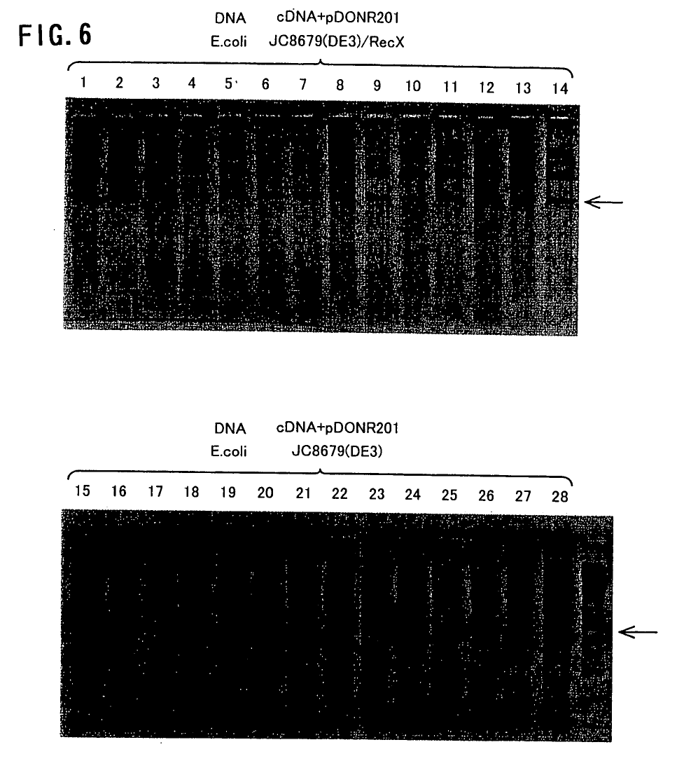

Next, transformation and further the incubation step were carried out basically in the same manner as in Examples 1 and 2. However, in this Example, the single colony incubation was carried out immediately after the plate incubation. And, in the same manner as in Examples 1 and 2, a plasmid DNA derived from each single colony was extracted (nucleic acid extraction step), and agarose gel electrophoresis was conducted (electrophoresis step), followed by recording on a photograph. The results are shown in FIG. 6.

Lane 1 to Lane 14 are the results obtained by conducting the above-described reaction, showing the results of electrophoresis for the plasmid DNA that was isolated and purified from each separate E. coli colony.

Lane 15 to Lane 28 are the results of a Comparative Example, in which E. coli JC8679 strain was used as it is as a host cell for transformation. That is, the E coli JC8679 strain without an expression vector which contains the RecX protein gene was used. Other procedures except this were the same as those in Lane 1 to Lane 14. Lane 15 to Lane 28 are also the results of electrophoresis for the plasmid DNA that was isolated and purified from each separate E. coli colony.

Further, the right lane of Lane 28 indicates the results of electrophoresis of a marker.

The signal seen at the position indicated by the arrow in FIG. 6 is the recombinant plasmid DNA in which the target region of the target nucleic acid (cDNA clone) was incorporated into the recombination vector (modified pDONR201 vector).

As is clear from the results of FIG. 6, in any of Lane 1 to Lane 14, which are the results for the Example, the recombinant plasmid DNA was obtained stably. Further, as far as it is shown in the results of electrophoresis, it can be inferred that undesired recombination does not occur. The signals seen above the signal at the position indicated by the arrow are considered to be multimers of the recombinant plasmid DNA, similarly to Examples 1 and 2.

On the other hand, also in Lane 15 to Lane 28, which are the results for the Comparative Example, the recombinant plasmid DNA was obtained stably. In addition, as far as seen from the results of electrophoresis, it is inferred that undesired recombination does not occur. However, the number of multimers of the recombinant plasmid DNA was much larger than that of the Example (Lane 1 to Lane 14).

From these results, it is found that the recombinant plasmid DNA is obtained stably by expressing the RecX protein by applying the present invention. It is also found that undesired recombination can be suppressed by expressing the RecX protein. Further, it is found that the number of the multimers of the recombinant plasmid DNA is reduced. On the other hand, it is found that when the RecX protein is not expressed, the number of the multimers of the recombinant plasmid DNA increases.

Further, not only in the Example but also in the Comparative Example, the reason for which undesired recombination does not occur, is considered to be that, since the single colony incubation is carried out immediately after the plate incubation, the recombinant plasmid DNA is present for a short time in the host cell, and thus undesired recombination has not yet occurred. In the Comparative Example, if the recombinant plasmid DNA is present for a long time in the host cell by, for example, extending the incubation time after the transformation, extending further the standing time after the plate incubation, etc., the recombinant plasmid DNA drops out of the host cell, or undesired recombination frequently occurs as shown in the above-mentioned Examples 1 and 2.

On the other hand, in the Example, even if the recombinant plasmid DNA is present for a long time in the host cell by, for example, extending the incubation time, extending the standing time after the incubation, etc., there is no dropout of the recombinant plasmid DNA, and the recombinant plasmid DNA can be obtained stably while the undesired recombination does not occur.

Further, for the parts similar to those of Examples 1 or 2, the same effects as those of Example 1 or 2 are obtained in this Example.

### EXAMPLE 4

Next, Example 4 will be explained. Explanation for the parts similar to those of Examples 1 to 3 will be omitted or simplified.

First, Escherichia coli JC8679 strain (DE3) was prepared by introducing a modified pET14b vector, to express a RecX protein in the same manner as in Examples 1 to 3. As described above, the E. coli JC8679 (DE3) is constituted to enable the expression and recombination of a RecA protein, and the expression of the RecX protein by induction with IPTG.

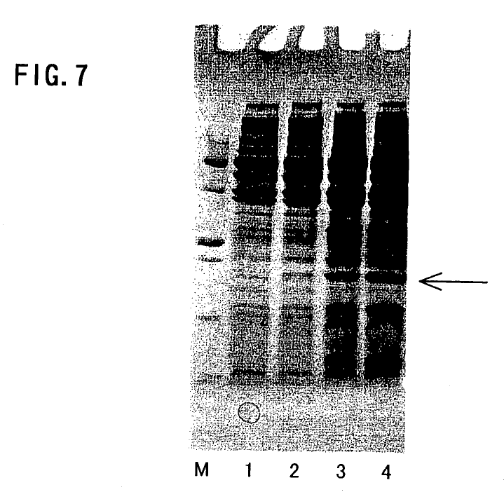

Next, on a 2 ml LB liquid medium to which ampicillin sodium and further 0.1 mM of IPTG were added, Escherichia coli JC8679 strain (DE3) with an expression vector was incubated overnight. Then, cells were collected by centrifuge and subsequently mixed with 100 μl of a cell-solubilizing solution, and the resultant solution was kept at 100° C. for 3 minutes. Then, an appropriate amount of the solution was subjected to electrophoresis with 12% SDS-PAGE, and the gel obtained after the electrophoresis was stained with CBB and recorded on a photograph. The results are shown in FIG. 7.

Lane 3 is the results obtained by conducting the above-described reaction.

Lane 4 is the results obtained by adding 0.5 mM of IPTG to the LB liquid medium while maintaining other procedures except this the same as those in Lane 3.

Lane 1 is the results of the Comparative Example, in which the E. coli JC8679 strain without the expression vector was used as it is. Other procedures except this were the same as those in Lane 3.

Lane 2 is the results also for the Comparative Example, in which the experiment was carried out with the E. coli JC8679 strain, into which the expression vector (pET14b vector) without the insertion of the RecX protein gene was introduced. Other procedures except this were the same as those in Lane 3.

Further, Lane M on the left side of Lane 1 is the results of electrophoresis for a marker.

The signal seen at the position indicated by the arrow in FIG. 7 shows the presence of the RecX protein.

As is clear from the results of FIG. 7, in Lane 3 and Lane 4 which are the results for the Example, expression of the RecX protein is observed. On the other hand, in Lane 1 in which the E. coli JC8679 strain without the expression vector was used, expression of the RecX protein was not observed. Also, in Lane 2 in which the E. coli JC8679 strain having the expression vector (pET14b vector) without the insertion of the RecX protein was used, expression of the RecX protein was not observed.

From these results, it is found that, with induction of the host cell by introducing the expression vector with the inserted RecX protein gene into E. coli, the RecX protein can be easily expressed. Accordingly, also in the above-mentioned Examples 1 to 3, it is found that when E. coli is induced, the RecX protein is expressed in the E. coli.

Further, for the parts similar to those of Examples 1 to 3, the same effects as those of Examples 1 to 3 are obtained in this Example.

In the above, the embodiments of the present invention were explained by Examples, but the present invention is not limited by the aforementioned Examples, and, needless to say, it can be suitably modified and applied without departing from the gist of the present invention.

What is claimed is:

1. A nucleic acid recombination method to carry out homologous recombination of nucleic acids in a host cell, which comprises
(a) a nucleic acid recombination step to carry out homologous recombination of the nucleic acids by a RecA-like protein expressed in the host cell, wherein the host cell is constituted to be able to express the RecA-like protein with ability of recombination and to express a RecX-like protein, and
(b) a RecA inhibition step to inhibit the activity of the RecA-like protein by expressing the RecX-like protein.

2. The nucleic acid recombination method according to claim 1, wherein
the host cell is prepared from a cell which originally has a RecA-like protein gene which expresses the RecA-like protein in its own genome.

3. The nucleic acid recombination method according to claim 1, wherein
the host cell has an expression vector into which a RecX-like protein gene is inserted, and in the RecA inhibition step, the RecX-like protein is expressed from the RecX-like protein gene.

4. The nucleic acid recombination method according to claim 3, wherein
the expression vector has an inducible promoter which induces the RecX-like protein gene, and in the RecA inhibition step, the RecX-like protein gene is induced by inducing the host cell to express the RecX-like protein.

5. The nucleic acid recombination method according to claim 1, wherein
in the nucleic acid recombination step, a vector which comprises a target nucleic acid containing a target region for recombination and a homologous recombination region capable of homologous recombination with the target region, are introduced into the host cell, and the target region is recombined into the recombination vector.

6. The nucleic acid recombination method according to claim 3, wherein the expression vector is composed of a pBR-based vector, and
in the nucleic acid recombination step, a target nucleic acid containing a target region for recombination, and a recombination vector which comprises a homologous recombination region capable of homologous recombination with the target region and is composed of a pUC-based vector, are introduced into the host cell, and the target region is recombined into the recombination vector.

7. The nucleic acid recombination method according to claim 1, wherein an incubation step to incubate the host cell is provided after the RecA inhibition step.

8. The nucleic acid recombination method according to claim 7, wherein
the expression vector has a first toxic substance resistant gene which gives resistance against a first toxic substance to the host cell, and the incubation step comprises a first incubation step to carry out incubation in the presence of the first toxic substance.

9. The nucleic acid recombination method according to claim 7, wherein
the recombination vector has a second toxic substance resistant gene which gives resistance against a second toxic substance to the host cell, and
the incubation step comprises a second incubation step to carry out incubation under the presence of the second toxic substance.

10. The nucleic acid recombination method according to claim 7, wherein
the incubation step comprises a plate incubation step to incubate the host cell on a plate medium to form a plurality of colonies on the plate medium, and a single colony incubation step to incubate separately each colony collected from the plate medium; wherein
the nucleic acid recombination method is provided with a nucleic acid extraction step to extract the nucleic acid which contains at least the recombinant vector from the host cell incubated by the incubation step with the single colony incubation step, and an electrophoresis step to electrophorese the extracted nucleic acid.

11. A host cell capable of homologous recombination of nucleic acids in its own cell, wherein
the host cell is constituted be able to express a RecA-like protein with ability of recombination and to express a RecX-like protein.

12. The host cell according to claim 11, wherein
the host cell is prepared from a cell which originally has a RecA-like protein gene expressing the RecA-like protein in its own genome.

13. The host cell according to claim 11, wherein
the host cell has an expression vector into which a RecX-like protein gene that expresses the RecX-like protein is inserted.

14. The host cell according to claim 13, wherein
the expression vector has an inducible promoter which induces the RecX-like protein gene, and the RecX-like protein gene is induced by inducing the host cell to express the RecX-like protein.

15. The host cell according to claim 13, wherein
the expression vector is composed of a pBR-based vector.

16. The host cell according to claim 13, wherein
the expression vector has a first toxic substance resistant gene which gives resistance against a first toxic substance to the host cell.

17. An expression vector which is introduced into a host cell and expresses a protein in the host cell, wherein
the expression vector has a RecX-like protein gene expressing a RecX-like protein.

18. The expression vector according to claim 17, wherein
the expression vector has an inducible promoter which induces the RecX-like protein gene and expresses the RecX-like protein.
